(19) United States
(12) Patent Application Publication
(10) Pub. No.: US 2006/0247530 A1
(43) Pub. Date: Nov. 2, 2006

(54) Echogenic markers on GI medical devices

(75) Inventors: David M. Hardin JR., Winston-Salem, NC (US); Kenneth C. Kennedy II, Clemmons, NC (US); Brian K. Rucker, King, NC (US); Gregory J. Skerven, Kernersville, NC (US)

Correspondence Address: BRINKS HOFER GILSON & LIONE, P.O. BOX 10395, CHICAGO, IL, 60610, US

(73) Assignee: Wilson-Cook Medical Inc., Winston-Salem, NC (US)

(21) Appl. No.: 11/365,580

(22) Filed: Feb. 28, 2006

Related U.S. Application Data
(60) Provisional application No. 60/657,540, filed on Feb. 28, 2005.

(51) Int. Cl. A61B 8/14

(52) U.S. Cl. 600/466

(57) ABSTRACT

An endoscopic ultrasound-guided system and method for monitoring the location of a device contained within intraluminal and extraluminal regions of a patient is described. The endoscopic ultrasound-guided system includes a linear echoendoscope, a device, and a wire guide. The device and wire guide contain echogenic surfaces which enable transducers placed at the distal end of the linear echoendoscope to ultrasonically monitor the location of the devices. When the echogenic surface of the device encounters incident ultrasound waves emitted from a series of linear array transducers, a real-time ultrasonic image of the device is generated as the incident ultrasound waves reflect off the echogenic surfaces and propagate back towards the transducers. The surgeon receives the real-time ultrasonic image of the device and then can determine the location of the device within the intraluminal or extraluminal region of the patient. After determining the location of the device, the surgeon can adjust the path of the device to ensure it is guided to the target site.

### RELATED APPLICATION

This application claims the benefit of priority from U.S. provisional application No. 60/657,540 filed Feb. 28, 2005, which is incorporated herein by reference.

### TECHNICAL FIELD

The invention generally relates to methods and systems for monitoring the location of a device within intraluminal and extraluminal regions of a patient.

### BACKGROUND

The ability to monitor the location and orientation of surgical instrumentation within intraluminal and extraluminal regions of a patient is critical. Fluoroscopy and radiopaque materials have traditionally been used to create visible regions of the digestive tract. Fluoroscopy is a technique in which an x-ray beam is transmitted through a patient to generate images of the gastrointestinal (GI) lumen that appear on a television monitor. It can also be used to observe the action of instruments during diagnostic procedures. However, x-rays consist of electromagnetic radiation which can be dangerous to the bile duct and pancreatic duct.

Conventional endoscopy offers visualization of the intraluminal regions through which the endoscope is inserted due to a video camera attached at the distal end of the endoscope. However, the video camera provides a field of view limited to only the intraluminal region. The use of surgical instrumentation outside of the lumen into extraluminal regions cannot be visualized with the endoscopic video camera.

Medical ultrasound has been another option used to monitor instrumentation. Medical ultrasound utilizes high frequency sound waves to create an image of living tissue. As ultrasound waves are emitted, the waves reflect when encountering a surface change. The reflected waves are used to create an image. However, conventional medical ultrasound has the drawback of ultrasound attenuation occurring in which a significant loss of energy occurs as the ultrasound waves pass through biological tissue. Consequently, poor images are created.

In view of the drawbacks of current technology, there is an unmet need to effectively monitor the real-time location, orientation, and depth of penetration of medical devices guided within intraluminal and extraluminal regions of a patient. Such monitoring is necessary to ensure medical devices are guided to their target sites and not inadvertently damaging adjacent tissue. Furthermore, the ability to perform such real-time monitoring of the devices will shorten surgical procedure times.

### SUMMARY

Accordingly, an endoscopic ultrasound (EUS)-guided device system is provided.

In one aspect, a system is disclosed for monitoring the location of a device within intraluminal and extraluminal regions. This is accomplished by an endoscopic ultrasound (EUS)-guided device system. The EUS-guided device system includes a linear echoendoscope and a device having an echogenic surface. The device contains a lumen adapted to receive a wire guide having an echogenic surface. Ultrasounds are emitted from transducers located at the distal end of the linear echoendoscope. The reflections of ultrasound waves from the echogenic surfaces of the wire guide and device enable a surgeon to precisely monitor the location of the wire guide and device within the lumen and extraluminal regions of a patient.

In a second aspect, a EUS-guided device system is disclosed for monitoring devices as they create access to extraluminal regions within a patient. The system includes a linear echoendoscope and a needle having a lumen and an echogenic surface. A wire guide having an echogenic surface coaxially fits within the lumen of the needle. Incorporation of echogenicity on the needle device and wire guide device enables a surgeon to precisely monitor the location of the devices as they are advanced to selected extraluminal regions in a patient and removed therefrom.

In a third aspect, a method for guiding a device in an intraluminal or extraluminal region is disclosed. The method includes positioning a linear echoendoscope within the lumen of a patient. The device is loaded coaxially through an accessory channel of the linear echoendoscope. Linear array transducers are activated. As the distal end of the device passes through the distal end of the accessory channel, the echogenic surface of the device encounters incident ultrasound waves emitted from a series of linear array transducers. A real-time ultrasonic image of the device is generated as the reflected ultrasound waves are detected by the transducers. The surgeon receives the real-time ultrasonic image of the device and then can determine the precise location of the device within the intraluminal or extraluminal region of a patient. After determining the location of the device, the surgeon can make any necessary adjustments to the location of the device to ensure the device is guided to the target site.

### BRIEF DESCRIPTION OF THE DRAWINGS

Embodiments of the present invention will now be described by way of example with reference to the accompanying drawings, in which:

FIG. 1 is a cross-sectional view of a linear echoscope advanced within a gastrointestinal lumen, having an unexpanded basket assembly loaded into the accessory channel of the linear echoscope;

FIG. 2 is a side view of an echogenic expandable basket assembly for retrieving foreign matter;

FIG. 3 is an elevational view of the echogenic expandable basket assembly of FIG. 2;

FIG. 4 is an elevational view of an echogenic wire guide;

FIG. 5 is a cross-sectional view of a linear echoscope advanced within a stomach, having an echogenic needle loaded into the accessory channel of the linear echoscope;

FIG. 6 is an partial cross-sectional view of a needle having an echogenic distal end;

FIG. 7 is an elevational view of an echogenic needle having three echogenic surfaces located at predetermined intervals along the distal end of the echogenic needle of the present invention;

FIG. 8 is an elevational view of the echogenic needle of FIG. 7 ultrasonically guided to a target pseudocyst;

FIG. 9 is an elevational view of the echogenic needle of FIG. 7 penetrating the target pseudocyst;

FIG. 10 is an elevational view of an echogenic stent having three echogenic surfaces spaced along distal end;

FIG. 11 is an elevational view of a needle knife having an echogenic distal end and an electrocautery wire disposed within a lumen of the needle knife;

FIG. 12 is a cross-sectional view of a linear echoscope advanced within a stomach, with the linear echoendoscope having an echogenic biopsy needle loaded into the accessory channel of the linear echoscope;

FIG. 13 is an elevational view of a biopsy needle having a catheter with an echogenic distal end;

FIG. 14 is an elevational view of a biopsy needle having a stylet with an echogenic distal end loaded into the catheter of FIG. 13;

FIG. 15 is an elevational view of a cytology brush having an echogenic distal end;

FIG. 16 is a perspective view of a gastrointestinal device having three circumferential echogenic surfaces at predetermined distances from each other; and

FIG. 17 is an elevational view of a gastrointestinal device having a smooth outer surface and an echogenic surface along an inner wall of the lumen of the gastrointestinal device.

### DETAILED DESCRIPTION OF THE PREFERRED EMBODIMENTS

The term echogenic refers to the extent that a surface reflects incident ultrasound wave energy directly back to a transducer or series of transducers. Enhanced echogenicity of a surface can be created by any technique that creates a surface indentation such that the dimensions of the surface indentation are substantially less than the incident ultrasonic sound waves. Intensity of the reflected and scattered waves is amplified by increasing the change in acoustic impedance between the surrounding medium (e.g., biological tissue) and the echogenic surface.

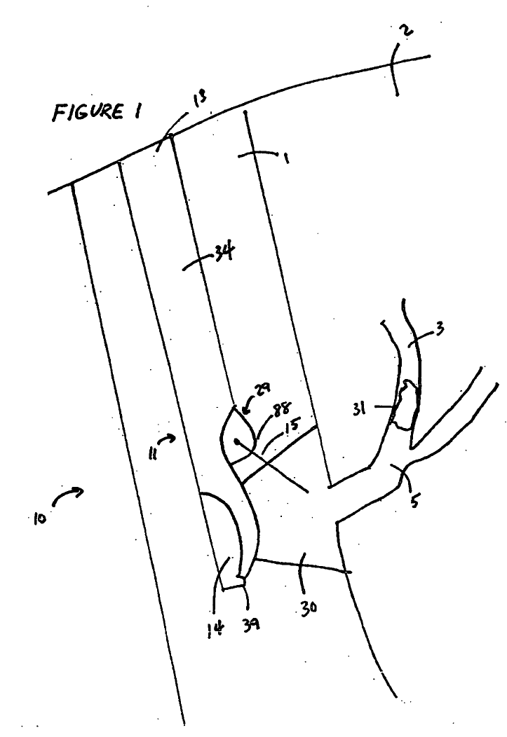

One embodiment of the present invention incorporates echogenicity into medical devices commonly used in endoscopic retrograde cholangiopancreatography (ERCP) to identify and retrieve gallstones or other foreign matter from the biliary and pancreatory ducts. By way of a non-limiting example, FIGS. 1-4 show endsoscopic ultrasound (EUS)-guided device system 10 capable of providing real-time information concerning the location and orientation of various echogenic devices utilized to effectively capture gallstone 31 lodged within biliary duct 3.

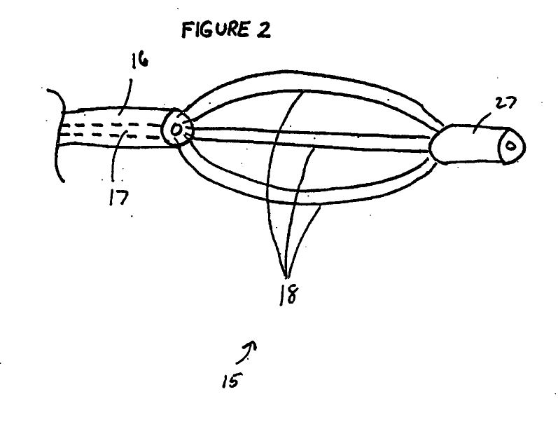

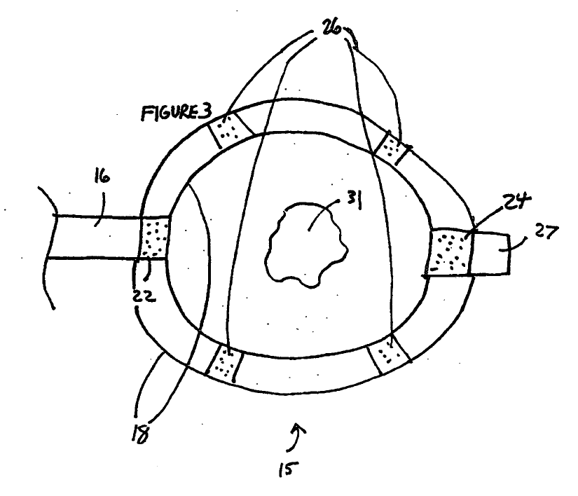

EUS-guided device system 10 comprises a linear echoendoscope 11 and a basket assembly 15 (shown in FIGS. 2-3). As shown in FIG. 1, linear echoendoscope 11 comprises a longitudinal shaft 34, a linear array of transducers 14 situated at the distal end 39 of linear echoendoscope 39, and an accessory channel 29. An unexpanded basket assembly 15 is loaded within the accessory channel 29. Transducers 14 generate an ultrasonic scanning plane 30. Placement of basket assembly 15 into the view of ultrasonic scanning plane 30 allows real-time monitoring of their respective locations and orientations within GI lumen 1, biliary duct 3 and extraluminal cavity 2. Such real-time monitoring may allow a variety of diagnostic and therapeutic maneuvers to be performed. Furthermore, because the linear transducers 14 emit ultrasound waves from within the GI lumen 1, substantially less attenuation of the ultrasound waves may occur as the ultrasound waves pass through tissue.

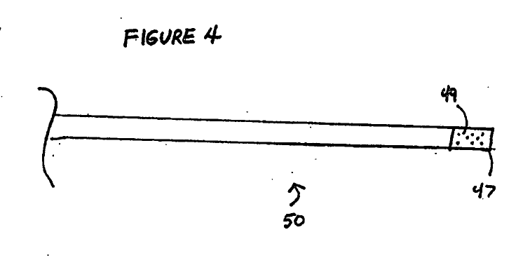

An elevational view of an echogenic wire guide 50 is shown in FIG. 4. Wire guide 50 comprises an echogenic surface 49 at the distal end 47.

A side view of the basket assembly 15 is shown in FIG. 2 and comprises multiple expandable arms 18 joined between the distal end of proximal flexible shaft 16 and the proximal end of distal flexible shaft 27. A lumen 17 is disposed within the proximal flexible shaft 16 and the distal flexible shaft 27 for insertion of wire guide 50 therethrough.

FIG. 3 indicates an elevational view of echogenic basket assembly 15 with arms 18 expanded around target gallstone 31. As will be explained in greater detail below, portions of the outer surfaces of basket assembly 15 may be surface treated to create the desired echogenicity. Providing selected portions of echogenic basket assembly 15 that are observable to the surgeon on a EUS display screen (not shown) provides the surgeon with the ability to effectively maneuver echogenic basket assembly 15 and ensare gallstone 31 for capture.

Multiple echogenic surfaces 26 along each of the arms, shown in FIG. 3, are provided for enhanced ultrasonic visualization of basket assembly 15 in relation to gallstone 31. Multiple echogenic surfaces 26 also ensure basket assembly 15 remains in the field of view of ultrasonic scanning plane 30 if incident ultrasound waves are inadvertently missing the distal-most echogenic surface 24 of basket assembly 15.

Providing echogenicity at the convergence of arms 18 at their distal end 24 and proximal end 22, as shown in FIG. 3, enables the surgeon to visualize where gallstone 31 is situated relative to the arms 18.

Referring back to FIG. 1, during ERCP, a surgeon advances linear echoendoscope 11 within GI lumen 1. The distal end of linear echoendoscope 11 is advanced as close as possible to papilla opening 5 and gallstone 31. At this point, gallstone stone 31 is readily observable due to its hyperechoic structures in which reflectance of incident ultrasound waves produce an image. With linear echoendoscope 11 deployed in a desired position, wire guide 50, shown in FIG. 4, is loaded from proximal end 13 of linear echoendoscope 11 and through accessory channel 29 of linear echoendoscope 11. When the distal end 47 of wire guide 50 has reached the distal end 88 of accessory channel 29, the surgeon turns on the linear array of transducers 14. The linear array of transducers 14 are sequentially activated via time delay circuits in such a manner that an ultrasonic scanning plane 30 is formed. The scanning plane 30 sweeps through wire guide 50 with a wedge-shaped geometry.

As the distal end 47 of wire guide 50 emerges from the distal end 88 of accessory channel 29 of linear echoendoscope 11, the surgeon is provided with visualization of echogenic surface 49. Ultrasound waves emitted from the linear array of transducers 14 are reflected from echogenic surface 49, thereby causing a sonographic image to appear on a EUS display panel (not shown). Because the linear array of transducers 14 are small enough to be located on the distal end 39 of linear echoendoscope 13, as shown in FIG. 1, incident ultrasound waves emitted from transducers 14 are required to propagate substantially less distance than if the transducers 14 were located external of the patient. The net effect of propagating less distance is that there is substantially less loss of energy as incident ultrasound waves emitted from linear array of transducers 14 travel through tissue and strike echogenic surface 49. Because the reflected waves may incur less loss of energy, the transducers 14 may detect the reflected waves and create an electrical signal of adequate intensity which in turn ensures the real-time image of wire guide 50 is discernable.

A real-time image is constructed from a series of small pixels on EUS display screen. Each dot represents a single reflected ultrasound pulse. The brightness of each pixel varies with the amount of reflected ultrasound energy. The location of the pixel represents the position of the reflecting interface. Consequently, on a EUS display screen, reflecting areas of high intensity appear white (hyperechoic) and areas of low reflection appear dark (hypoechoic). Such enhanced ultrasonic visualization will allow the surgeon to precisely navigate wire guide 50 through papilla opening 5 and into biliary duct 3 towards gallstone 31. Because gallstone 31 is hyperechoic, the surgeon will be able to continuously monitor the location of echogenic wire guide 50 in relation to gallstone 31. The entire path of wire guide 50 towards gallstone 31 may be visualized.

After the surgeon has positioned wire guide 50 into biliary tract 3 and in close proximity to gallstone 3 1, basket assembly 15 can be loaded into accessory channel 29 coaxially over wire guide 50, which serves as a stable guide to facilitate deployment of basket assembly 15 into biliary duct 3. FIG. 1 illustrates basket assembly 15 completely loaded into accessory channel 29 with arms 18 unexpanded and ready for deployment into GI lumen 1, through papilla opening 5, and into biliary tract 3 where gallstone 31 is lodged therewithin. Echogenic surfaces 22, 24, and 26 of basket assembly 15 will enable the surgeon to visualize the location and orientation of the basket assembly 15 as it is guided towards gallstone 3 1.

The ability to observe the positions of both gallstone 31 and basket assembly 15 significantly reduces the amount of time a surgeon must expend within biliary duct 3. Such a reduction in procedure time also mitigates patient trauma and potential injury to bile duct 3 due to inadvertent puncture of adjacent tissue.

Although the above procedure has been described with the echogenic basket assembly 15 mounted onto an echogenic wire guide 50, the embodiment also contemplates navigation of the basket assembly 15 without any wire guide. Furthermore, the embodiment also contemplates various other medical devices having echogenic surfaces which may be used with or without a wire guide.

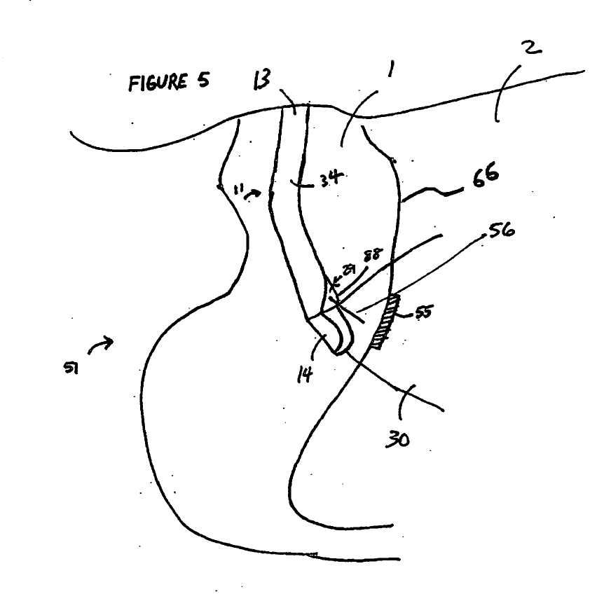

In accordance with another embodiment of this invention, echogenicity can also be incorporated on a variety of GI devices to perform procedures in the extraluminal regions. By way of a non-limiting example, FIGS. 4-11 illustrate another embodiment of this invention in which a particular EUS-guided device system 5 1, shown in FIG. 5, can be used to effectively drain a pseudocyst 55 growing on the bottom of stomach wall 66.

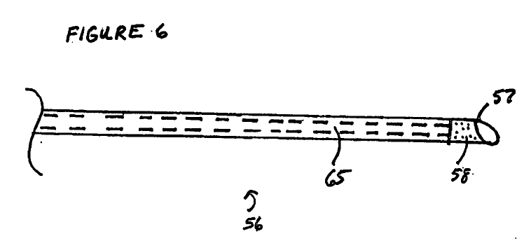

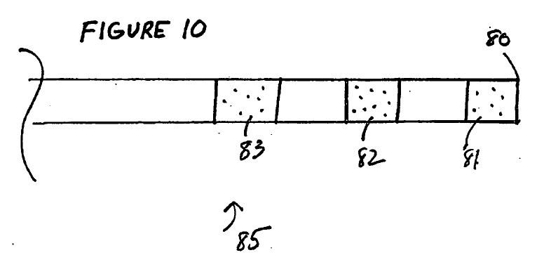

EUS-guided device system 51 comprises linear echoendoscope 11, needle 56 (as shown in FIG. 6), and one or more stents 85 (as shown in FIG. 10). As shown in FIG. 5, linear echoendoscope 11 comprises a longitudinal shaft 34, a linear array of transducers 14 for generating a ultrasonic scanning plane 30, and accessory channel 29 for advancing various echogenic GI medical devices therethrough. Transducers 14 generate ultrasonic scanning plane 30, as shown in FIG. 5. The placement of wire guide 50, needle 56, and stents 85 into the ultrasonic scanning plane 30 of view allows real-time monitoring of their respective locations and orientation within GI lumen I and extraluminal region 2, thereby permitting a variety of diagnostic and therapeutic maneuvers to be performed.

Needle 56, shown in FIG. 6, has an outer echogenic surface 58 located about distal end 57. Needle 56 may also includes a lumen 65 for receiving wire guide 50.

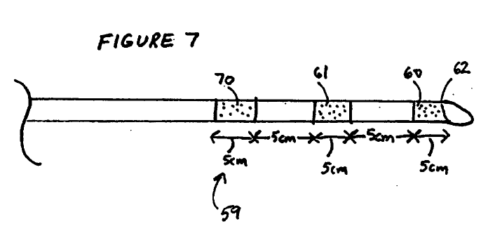

Although needle 56 is illustrated to have only one echogenic surface 58, multiple echogenic surfaces can also be used. Multiple echogenic surfaces that are spaced apart at predetermined distances can permit greater determination of the location and orientation of a EUS-guided needle. As an example, FIG. 7 illustrates three echogenic surfaces along distal end 62 of needle 59. In particular, needle 59 has echogenic surface or band 60 positioned about distal end 62, echogenic surface or band 61 positioned 5 cm proximal to echogenic surface 60, and echogenic surface or band 70 positioned 5 cm proximal to echogenic surface 61. Echogenic surfaces 60, 61, and 70 each have a longitudinal dimension of 5 cm as shown in FIG. 7. Having multiple echogenic surfaces 60, 61, and 70 spaced at predetermined distances from each other enhances the surgeon's monitoring of the location of the needle relative to pseudocyst 55. It also ensures that needle 59 remains in the field of view of ultrasonic scanning plane 30 if incident ultrasound waves inadvertently do not strike the distal-most echogenic surface 60.

Echogenic surfaces 60, 61, and 70 also provide the ability to monitor the orientation of needle 59. Three distinct echogenic regions on needle 59 will generate three distinct white pixels on the EUS display panel (not shown) when echogenic surfaces 60, 61, and 70 are within the field of view of ultrasonic scanning plane 30. The relative vertical and horizontal orientation of the three pixels on the EUS display panel corresponds to the orientation of needle 59 within extraluminal region 2. Such real-time information can be used by the surgeon to determine whether the distal end 62 of needle 59 is in proper orientation to make the desired puncture upon reaching stomach wall 66. If needle 59 is not in its proper orientation, then the surgeon will know to remaneuver needle 59 accordingly until the desired orientation appears on the EUS display panel.

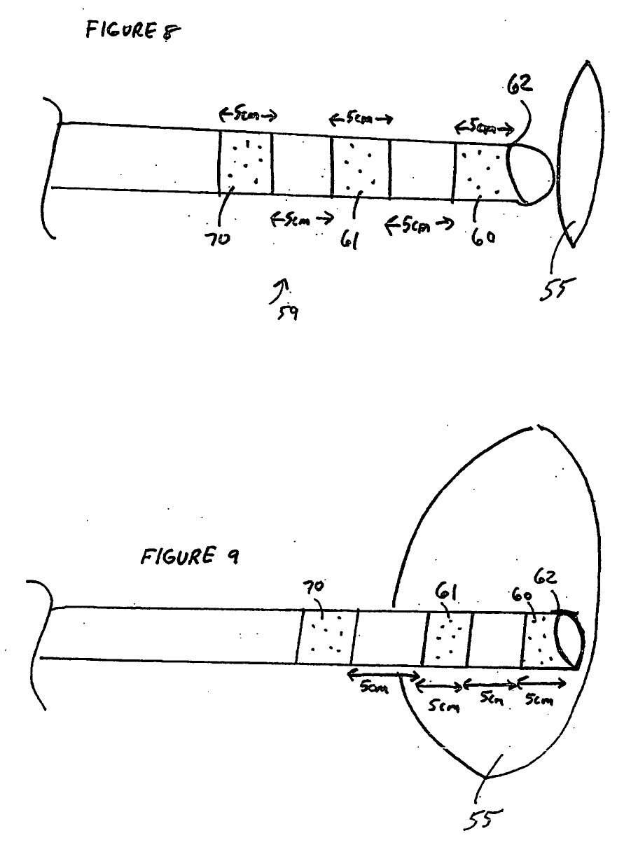

Additionally, multiple distinct regions of echogenicity on needle 59 may also convey depth of penetration of needle 59 into pseudocyst 55. FIG. 8 depicts EUS-guided needle 59 advancing towards the target pseudocyst 55. Echogenic surfaces 60, 61, and 70 may create enhanced visualization of needle 59 advancing in close proximity to pseudocyst 55. As the surgeon proceeds to make the desired puncture into pseudocyst 55, the predetermined spacings of echogenic surfaces 60, 61, and 70 may indicate the depth of penetration of needle 59 into pseudocyst mass 55. For example, in FIG. 9, the distinct separation of echogenic region 70 from pseudocyst 55 on a EUS display panel may indicate to the surgeon that needle 59 has penetrated at least 15 cm but not more than 20 cm into pseudocyst mass 55. Obtaining such real-time information from the echogenicity of needle 59 is critical for knowing whether access has been obtained and, thereafter, whether successful incision into pseudocyst mass 55 has been created.

Referring back to FIG. 5, after linear echoendoscope 11 is advanced in close proximity to pseudocyst 55, needle 56 is loaded at proximal end 13 of linear echoendoscope 11. Needle 56 is deployed through accessory channel 29 of linear echoendoscope 11 for the purpose of puncturing stomach wall 66 to access the desired extraluminal location of pseudocyst 55. FIG. 5 depicts needle 56 fully loaded into the distal end 88 of accessory channel 29 and ready for deployment into GI lumen 1, towards the portion of stomach wall 66 containing pseudocyst 55.

At this stage, the surgeon turns on linear array transducers 14, located at the distal tip of linear echoendoscope 11. Transducers 14 are sequentially activated via time delay circuits in such a manner that a wedge-shaped ultrasonic scanning plane 30 encompasses needle 56. Because ultrasonic scanning plane 30 is parallel to longitudinal shaft 34, the entire path of needle 56 to stomach wall 66 can be followed as echogenic surface 58, shown in FIG. 6, emerges out of the distal end 88 of accessory channel 29.

After needle 56 has created access into pseudocyst 55, wire guide 50 is loaded through the proximal end 13 of linear echoendoscope 11 coaxially into the lumen 65 of needle 56. As the distal end 47 of wire guide 50 emerges from accessory channel 29, visualization of the path of wire guide 50 through punctured pseudocyst 55 may be monitored as ultrasound waves emitted from linear array transducers 14 are reflected back from echogenic surface 49 towards transducers 14 thereby causing a sonographic image to appear on a EUS display panel (not shown). The entire path of wire guide 50 towards stomach wall 66 may be followed as echogenic surface 49 portion emerges out of the distal end 88 of accessory channel 29 towards the puncture site of pseudocyst 55.

With wire guide 50 maintaining access at the puncture site of pseudocyst 55, needle 56 can be withdrawn. Accordingly, needle 56 is withdrawn from pseudocyst 55 and back into accessory channel 29, and upwards through longitudinal shaft 34 of linear echoendoscope 11. The surgeon may accurately monitor withdrawal of needle 56 as ultrasound imaging provides real-time information concerning the location of distal echogenic surface 58 of needle 56.

Echogenic wire guide 50 may now act as a stable guide. Several stents 85, each as shown in FIG. 10, are sequentially loaded coaxially onto wire guide 50. Stents 85 are used to further dilate pseudocyst 55 thereby facilitating quicker drainage of its contents into the stomach lumen 1.

FIG. 10 illustrates a strut of one of the stents 85 that may be utilized. Stent 85 has three echogenic surfaces 81, 82, 83 at predetermined intervals along its distal end 80. Echogenic surfaces at predetermined distances along distal end 80 of stent 85 may enable the surgeon to determine the depth of penetration of stent 85 into pseudocyst 55. Ultrasonic imaging is also facilitated by stent 85 containing multiple surfaces. Multiple echogenic surfaces 81, 82, 83 provide additional visible regions when incident ultrasonic waves are not reflecting off the distal-most echogenic surface 81 of stent 85. Such additional visible regions assure that stent 85 remains in the field of view of ultrasonic scanning plane 30.

Deploying stent 85 into the hole of pseudocyst 55 may include the following steps. The surgeon first advances distal end 80 of stent 85 into the accessory channel 29 of linear echoendoscope 11. As the distal end 80 emerges from the distal end 88 of accessory channel 29, a ultrasonic scanning plane 30 is generated by linear array transducers 14. Ultrasound waves emitted from linear array transducers 14 are reflected back from echogenic surfaces 81, 82, 83 to transducers 14. Linear array transducers 14 detect the reflected waves and translate the waves back into electrical signals for processing into an image on the EUS diplay monitor (not shown). Because ultrasonic scanning plane 30 is parallel to longitudinal shaft 34, the entire path of stent 85 to pseudocyst 55 can be followed via ultrasonic visualization of echogenic surfaces 81, 82, 83.

The ability for a surgeon to continuously monitor real-time location and orientation of the path of stent 85 may allow the surgeon to make adjustments to the path of stent 85, if necessary. Such adjustments may help avoid damage to adjacent tissue and help deploy stent 85 with optimal orientation into pseudocyst 55. Multiple echogenic surfaces 81, 82, 83 may also serve to enhance ultrasonic visualization during deployment of stents 85 by assuring stents 85 remain in the field of view of ultrasonic scanning plane 30 if incident ultrasound waves inadvertently miss reflecting off the distal-most echogenic surface 81 of stent 85. Moreover, echogenic surfaces 81, 82, 83 provide the surgeon information regarding depth of penetration of stent 85 into pseudocyst 55. Such precise echogenic guiding may allow the surgeon to deploy multiple stents 85 to further dilate hole of pseudocyst 55 for quicker drainage, which in turn may lead to faster recovery times.

In accordance with another embodiment of the present invention, echogenic technology allows traditional intraluminal devices to also be used to gain access to extraluminal regions. As a non-limiting example, needle knives of the type commonly used to access the bile duct 3 may be modified to incorporate echogenicity to the distal portion thereof to expand its applications to access extraluminal regions.

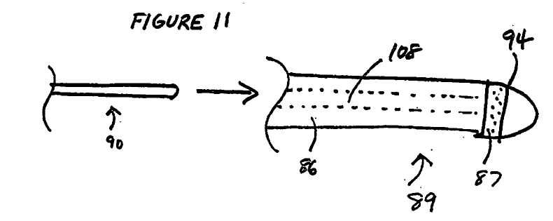

FIG. 11 illustrates a needle knife 89 having plastic outer protective sleeve 86 with echogenic surface 87 about distal end 94 and a lumen 108 through which thin electrocautery wire 90 is inserted. Needle knife 89 may now be used to access pseudocyst 55 and burn peripheral tissue of the pseudocyst 55 to potentially facilitate quicker drainage of the pseudocyst 55 contents.

Referring back to the method for drainage of pseudocyst 55 depicted in FIG. 5, after needle 56 has been removed from the puncture site it created in pseudocyst 55, shown in FIG. 9, needle knife 89 can be introduced into accessory channel 29 of linear echoendoscope 11 and advanced coaxially over wire guide 50 to the puncture site of pseudocyst 55. Needle knife 89 is guided to pseudocyst 55 by ultrasonically monitoring the location of echogenic distal end 87 of needle knife 89. After positioning wire knife 89 in proximity to pseudocyst 55, thin electrocautery wire 90, disposed within lumen 108, can be used to heat and burn peripheral tissue of pseudocyst 55, thereby dilating the puncture of pseudocyst 55 initially created by needle 56.

As an alternative to having one echogenic distal end 87 as shown in FIG. 11, it should be understood that multiple echogenic surfaces can be provided about or near distal tip 94 of plastic outer protective sleeve 86. This will enable the surgeon to determine vertical and horizontal orientation of needle knife 89, and the depth of penetration of needle knife 89 into pseudocyst 55. Preferably, maintaining a constant depth of penetration during heating of peripheral tissue by electrocautery wire 90 can ensure there is no tearing or unnecessary trauma to adjacent wall tissue.

After dilation of the hole is completed by electrocautery wire 90, one or more stents 85, as shown in FIG. 10, are ultrasonically guided into the dilated hole of pseudocyst 55 by monitoring the echogenic surfaces 81, 82, 83 along distal end 80. Stents 85 will maintain the dilation thereby facilitating drainage of the contents of pseudocyst 55.

In accordance with another embodiment of the present invention, incorporation of echogenicity to GI accessories can significantly enhance EUS-guided fine-needle aspiration (FNA) biopsies of mucosal and submucosal lesions, peri-intestinal structures including lymph nodes, as well as masses arising in the pancreas, liver, adrenal gland, and bile duct. FIG. 4 and FIGS. 12-14 illustrate application of EUS-guided device system 95, shown in FIG. 12, to aspirate fluid from mass 96 on the bottom of the pancreas 97. After a scan has detected mass 96, a surgeon may maneuver in close proximity to mass 96 utilizing EUS-guided device system 95 to obtain an adequate sample of mass 96 to determine if it is cancerous.

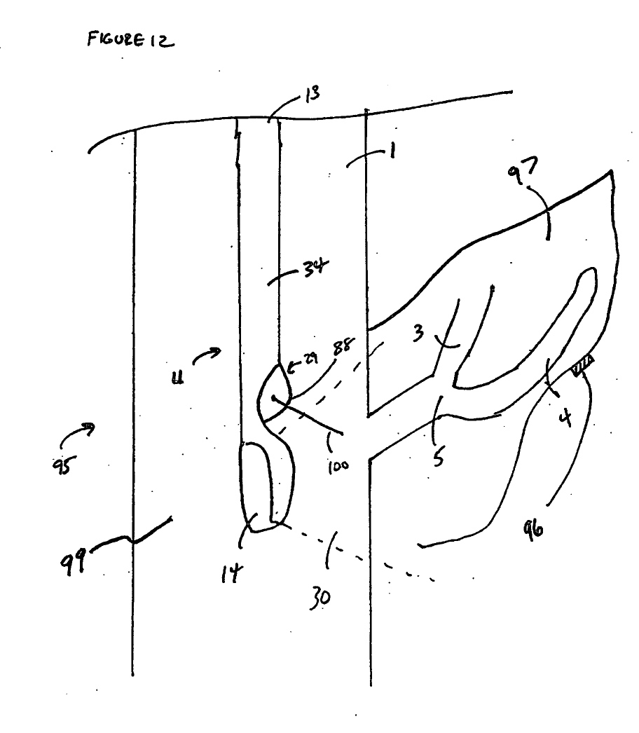

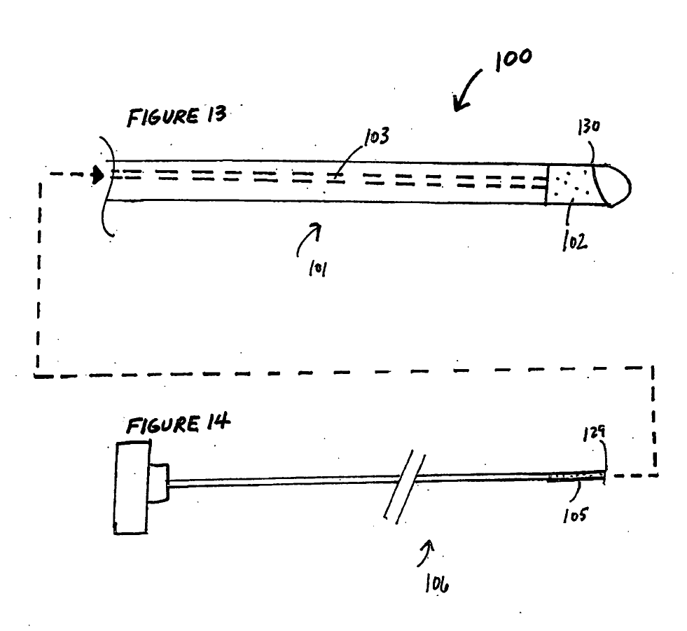

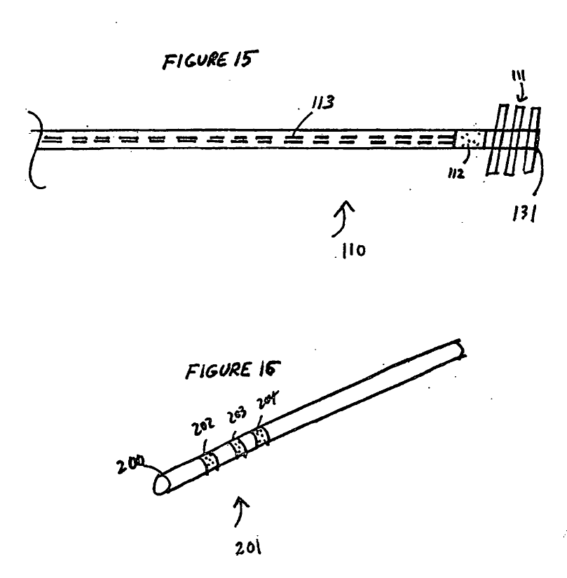

EUS-guided device system 95 comprises linear echoendoscope 11, needle 100, and cytology brush 110. Biopsy needle 100 is illustrated in FIGS. 13 and 14. Biopsy needle 100 comprises catheter 101, shown in FIG. 13, and stylet 106, shown in FIG. 14. Catheter 101 comprises lumen 103 and an echogenic surface 102 about distal end 130. Stylet 106 comprises an echogenic surface 105 about distal end 129. Stylet 106 is loaded into the lumen 103 of catheter 101. Cytology brush 110 is illustrated in FIG. 15 and comprises echogenic surface 112 about distal end 131, bristles 111, and a lumen 113 adapted to receive wire guide 50.

FIG. 12 illustrates a method for EUS-guided fine-needle aspiration biopsies (FNA). Staying within GI lumen 1, linear echoendoscope 11 is advanced down through the esophagus and into duodenum 99. Linear echoendoscope 11 is maneuvered by the surgeon as close as possible to papilla 5. Next, biopsy needle 100 is loaded into the proximal end 13 of linear echoendoscope 11 through accessory channel 29. Linear array transducers 14 are turned on. As the distal end 130 of biopsy needle 100 emerges from the distal end 88 of accessory channel 29, visualization of the path of biopsy needle 100 relative to mass 96 can be monitored. Ultrasonic sound waves emitted from linear array transducers 14 are reflected from echogenic surface 102 back towards the linear array transducers 14, thereby causing a sonographic image to appear on a EUS display panel (not shown). Because ultrasonic scanning plane 30 is parallel to longitudinal shaft 34, as shown in FIG. 12, the entire path of biopsy needle 100 towards mass 96 may be within the field of view of the ultrasonic scanning plane 30.

As an alternative to having one echogenic surface 102 about distal end 130 of catheter 101, it should be understood that multiple echogenic surfaces about distal end 131 may also be added to determine vertical and horizontal orientation of needle knife 89 and the depth of penetration of needle knife 89. Furthermore, multiple echogenic surfaces positioned proximal to echogenic surface 102 provide additional visible regions when incident ultrasound waves are not capable of reflecting off the distal-most echogenic surface 102 of catheter 101. Such additional visible regions may assure that catheter 101 remains in the field of view of ultrasonic scanning plane 30.

After biopsy needle 100 has been precisely guided to mass 96, the surgeon may puncture mass 96 with swift back and forth movements of the biopsy needle 100 until distal end 130 has entered mass 96. Upon successful insertion of distal end 130 into mass 96, stylet 106 may be removed. The path of stylet 106 during its removal can be monitored by ultrasound waves reflecting off echogenic surface 105. As an alternative to one echogenic distal region, multiple echogenic surfaces about distal end 129 may be employed to enable the surgeon to determine the vertical and horizontal orientation of stylet 106 as it is guided towards the distal end 88 of accessory channel 29.

Aspiration of the contents from mass 96 includes applying negative pressure with a vacuum locking syringe (not shown) placed over or otherwise connected to the proximal end of catheter 101. Multiple to and fro movements of catheter 101 may be required to gain an adequate sample. At this point in the procedure, the surgeon monitors the relative location of echogenic surface 102 in relation to mass 96. Failure to monitor the location of catheter 101 may result in inadvertent withdrawal of catheter 101 outside of mass 96 during aspiration and into the intestinal lumen where mass 96 can be contaminated by luminal contents and the epithelium. The reflectance of ultrasound waves from echogenic surface 102 back towards linear array transducers 14 will enable the surgeon to monitor the real-time location of biopsy needle 100 during aspiration and avoid unintended movement of biopsy needle 100 into the intestinal lumen.

If the surgeon is not able to aspirate mass 96, then cytology brush 110 can be used to partially liquidate mass 96 with bristles 111. Wire guide 50 may be loaded through accessory channel 29 and thereafter navigated towards catheter 101 and into lumen 103 of catheter 101. As wire guide 50 emerges from the distal end 88 of accessory channel 29 into the GI lumen 1 (see FIG. 12), ultrasound waves emitted from linear array of transducers 14 are reflected from echogenic surface 49 (see FIG. 4) towards the transducers 14, thereby causing echogenic surface 49 to appear as a sonographic image on a EUS display panel. Because echogenic surface 102 of catheter 101 will be within the field of view of ultrasonic scanning plane 30, the surgeon will be able to visualize both the wire guide 50 and catheter 101 when guiding wire guide 50 into the lumen 103 of catheter 101.

With wire guide 50 loaded into lumen 103, the catheter 101 component of the biopsy needle 100 can be removed. The location of catheter 101 during its removal can be precisely controlled by monitoring the location of echogenic surface 102. As an alternative to the one echogenic surface 102 shown in FIG. 13, multiple echogenic surfaces about distal end 130 of catheter 101 can be utilized to enable the surgeon to determine the vertical and horizontal orientation of catheter 101 as the surgeon is maneuvering catheter 101 towards the distal end 88 of accessory channel 29.

After biopsy needle 100 has been removed, cytology brush 110 can now be inserted through linear echoendoscope 11 and into accessory channel 29. Wire guide 50 may act as a stable guide when disposed within the lumen 113 of cytology brush 10. As cytology brush 110 emerges from the distal end 88 of accessory channel 29 and begins its path towards mass 96, echogenic surface 112 will provide a visual marker the surgeon may use to achieve controlled ultrasound- guided maneuvering. Upon reaching mass 96, bristles 111 can be used to gradually blunder mass 96 until it partially liquidates. When mass 96 has been sufficiently blundered, cytology brush 110 is withdrawn from mass 96 and catheter 101 is reintroduced for aspiration. Visualization of echogenic surface 102 of catheter 101 and echogenic surface 112 of cytology brush may provide precise maneuvering and orientation thereby assuring a rapid exchange of the two devices. Such visualization may also provide a safe exchange of the two devices due to reduction of risk of inadvertent damage to surrounding tissue.

One of ordinary skill would realize that the above described EUS-guided device system 95 and method of uses thereof can also be used to inject seeds and other therapeutic agents into targeted extraluminal regions.

The above embodiments describing the EUS-guided device systems contemplate using the echogenic devices with or without a wire guide or echogenic wire guide.

One of ordinary skill would recognize that there are multiple obvious variations of echogenic surfaces on devices that can be utilized in accordance with all of the disclosed embodiments of the present invention. As an alternative to having only the top surface of a device echogenic, one of ordinary skill would realize that all of the described echogenic devices can have a circumferential echogenic band about the distal end to facilitate enhanced ultrasonic visualization. FIG. 16 illustrates a GI medical device 201 having three echogenic circumferential surfaces 202, 203, 204 evenly spaced about distal end 200. The echogenic circumferential surfaces extend three hundred sixty degrees along the outer surface of GI medical device 201. Such circumferential surfaces can increase the amount of incident ultrasound waves reflected off the GI medical device 201 thereby enhancing ultrasonic visualization of the device 201.

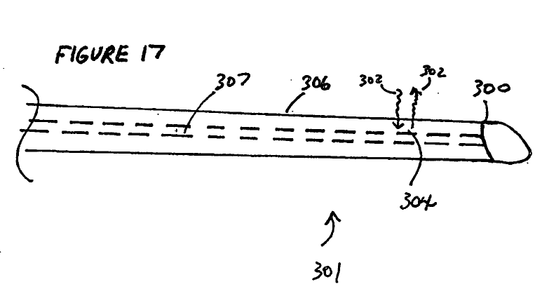

Additionally, to reduce trauma, devices containing lumens can utilize their inner surface walls as the echogenic surface thereby allowing a smooth outer wall that eliminates tissue trauma associated with movement of devices with echogenic outer surface indentations. FIG. 17 depicts a GI medical device 301 having echogenic inner surface 304 created on the wall of lumen 307. Incident ultrasound wave 302 would pass through smooth outer surface 306. Upon reaching echogenic inner surface 304, the ultrasound wave 302 is reflected back towards linear array transducers 14 (not shown). No attenuation of the ultrasound wave 302 occurs.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims. For example, the invention has been described in the context of accessing the biliary and pancreatic ducts, stomach wall, and pancreas. Application of the principles of the invention to access other body cavities, such as the thoracic cavity, by way of a non-limiting example, are within the ordinary skill in the art and are intended to be encompassed within the scope of the attached claims. Moreover, in view of the present disclosure, a wide variety of EUS guided device systems and methods of their uses will become apparent to one of ordinary skill in the art.

1. An endoscopic ultrasound-guided device system for monitoring the location of a device contained within a lumen or extraluminal region of a patient, the device comprising a proximal end and a distal end, wherein the distal end has an echogenic surface that is capable of being visually monitored, further wherein a longitudinal dimension of the device is sufficient to extend into a gastrointestinal tract of the patient.

2. The system of claim 1 wherein the device is selected from the group consisting of an expandable basket assembly, a stent, a cytology brush, a needle knife, a biopsy needle, and a catheter.

3. The system of claim 1 wherein the echogenic surface extends circumferentially around the distal end of the device.

4. The system of claim 1 wherein the echogenic surface is located on an inner surface of a wall of the lumen.

5. The system of claim 1, wherein the device has a lumen adapted for receiving an echogenic wire guide.

6. An endoscopic ultrasound-guided biopsy needle system for establishing access to extraluminal regions within a patient comprising:
a catheter further comprising a proximal end, a distal end, an echogenic surface about the distal end, and a lumen extending between the proximal and the distal end, the catheter having a longitudinal dimension that is sufficient to extend into a gastrointestinal tract of the patient; and
a stylet having a proximal end, a distal end, and an echogenic surface about the distal end, wherein the stylet is coaxially loaded into the lumen of the catheter.

7. The system of claim 6 further comprising a cytology brush having a proximal end, a distal end, an echogenic surface about the distal end, a lumen extending longitudinally between the proximal end and the distal end, and a plurality of bristles about the distal end.

8. A method for ultrasonically guiding a device in an intraluminal or extraluminal region of a patient comprising the steps of:
(a) positioning a linear echoendoscope within the intraluminal region, wherein the linear echoendoscope comprises a series of linear array transducers and an accessory channel having a distal end and a proximal end;
(b) loading a device through the proximal end of the accessory channel;
(c) activating the series of linear array transducers, wherein the linear array transducers emit a pulse of ultrasound waves;
(d) directing the emitted ultrasound waves onto an echogenic surface located along a distal end of the device as the device emerges from the distal end of the accessory channel into the intraluminal region;
(e) generating an ultrasonic image of the device from the pulse of ultrasound waves reflected from the echogenic surface along the distal end of the device, wherein the linear array transducers detect the reflected pulse of ultrasound waves and translate the ultrasound waves into electrical signals for processing into a real-time ultrasonic image; and
(f) determining a location of the device within the intraluminal or extraluminal region from the real-time ultrasonic image.

9. The method of claim 8 wherein the device is a wire guide.

10. The method of claim 8 wherein the device is a needle.

11. The method of claim 8 wherein the device is a stent.

12. The method of claim 8 wherein the device is a basket assembly.

13. The method of claim 8 wherein the device is a cytology brush.

14. The method of claim 8 wherein the device is a needle knife.

15. The method of claim 8 wherein the echogenic surface of the device extends circumferentially around the distal end.

16. The method of claim 8 wherein the echogenic surface of the device is located on an inner surface of a wall of a lumen of the device.

17. The method of claim 8 wherein determination of an orientation of the device further comprises the steps of:
(g) directing emitted ultrasound waves onto a device having at least two echogenic surfaces selectively placed at predetermined distances from each other along a distal end of the device;
(h) generating an ultrasonic image of the device from the pulse of ultrasound waves reflected from the at least two echogenic surfaces, wherein the linear array transducers detect the reflected pulse of ultrasound waves and translate the ultrasound waves into electrical signals for processing into real-time ultrasonic images;
(i) receiving the real-time ultrasonic images of the at least two echogenic surfaces; and
(j) determining an orientation of the device from the real-time ultrasonic images.

18. The method of claim 8 wherein determination of a depth of penetration of the device into biological tissue further comprises the steps of:
(g) penetrating biological tissue with the device having at least two echogenic surfaces spaced at predetermined distances along the distal end of the device; and
(h) ultrasonically monitoring locations of the two or more echogenic surfaces relative to the biological tissue.

19. The method of claim 17 wherein the at least two echogenic surfaces comprises at least one hyperechoic surface adjacent to a hypoechoic surface.

20. The method of claim 18 wherein the at least two echogenic surfaces comprises at least one hyperechoic surface adjacent to a hypoechoic surface.
